(19) United States
(12) Patent Application Publication
(10) Pub. No.: US 2026/0297318 A1
(43) Pub. Date: Oct. 1, 2026

(54) ANTI-SWELLING AND ANTI-DRYING HYDROGEL AND ELECTRODES WITH ZERO-GAUGE FACTOR CHARACTERISTICS INCLUDING THE SAME

(71) Applicant: KOREA ADVANCED INSTITUTE OF SCIENCE AND TECHNOLOGY, Daejeon (KR)

(72) Inventors: Steve Park, Daejeon (KR); Byung Kook Oh, Daejeon (KR); Soo Min Kim, Daejeon (KR)

(21) Appl. No.: 19/296,225

(22) Filed: Aug. 11, 2025

(30) Foreign Application Priority Data
Mar. 31, 2025 (KR) 10-2025-0040892

(51) Int. Cl. C08L 33/08; C08K 5/053; G01N 33/48; H01M 4/02; H01M 4/36; H01M 4/60

(52) U.S. Cl. CPC C08L 33/08; C08K 5/053; G01N 33/48; H01M 4/366; H01M 4/604; C08L 2203/20; C08L 2312/00; H01M 2004/023

(57) ABSTRACT

The present disclosure provides and anti-swelling and anti-drying hydrogel including a hydrogel matrix; and two types of micelle-crosslinked polymers formed within the hydrogel, wherein the two types of micelle-crosslinked polymers have different lengths, and micelle-crosslinked polymer with the shortest chain length functions to lower the glass transition temperature (Tg) of the micelle-crosslinked polymers.

### CROSS-REFERENCE TO RELATED APPLICATION

This application claims priority of Korean Patent Application No. 10-2025-0040892, filed on Mar. 31, 2025, in the KIPO (Korean Intellectual Property Office), the disclosure of which is incorporated herein entirely by reference.

### BACKGROUND OF THE INVENTION

### Field of the Invention

The present invention relates to an anti-swelling and anti-drying hydrogel and electrodes with zero-gauge factor characteristics including the same.

### Description of the Related Art

Hydrogels are extensively used as accessory materials in diagnostic instruments in the medical industry. These hydrogels are used in medical device electrodes such as ECG, EEG, EMG, TENS, and ESU grounding electrodes, and serve as materials that enhance the transmission of bioelectrical signals.

In particular, as the world enters a super-aged society, the incidence of intractable and degenerative diseases is increasing, leading to greater medical and economic burdens. In this context, hydrogel-based electrode materials are essential as next-generation electronic materials enabling real-time health monitoring.

Currently, hydrogels exhibit physical properties similar to skin and biological tissues, and offer excellent breathability and user comfort due to their high water content and porous structure. However, during use in daily life, water may infiltrate and dilute the hydrophilic network inside the hydrogel, leading to deformation (swelling) and mechanical degradation. In some cases, water evaporates in air, causing the material to shrink and harden.

Furthermore, electrodes for monitoring bio-signals in dynamic environments often involve modifications in electrode geometry or lamination onto soft substrates to improve mechanical durability. However, these approaches may result in high electrical resistance and complex fabrication processes, thereby hindering high-resolution monitoring.

It is thus necessary to minimize hydrogel swelling due to water or ion absorption. Therefore, there is an ongoing demand for the development of hydrogels that resist swelling and drying even without hydrophobic polymer coatings, and for electrode materials based on such hydrogels.

### SUMMARY OF THE INVENTION

Accordingly, an object of the present invention is to provide a hydrogel with both anti-swelling and anti-drying characteristics without the need for additional coatings, as well as electrode materials based on this hydrogel.

To achieve the above objective, the present invention provides an anti-swelling and anti-drying hydrogel comprising: a hydrogel matrix; and two types of micellar crosslinking polymers with different chain lengths formed in the hydrogel matrix.

In one embodiment, the shorter micellar crosslinking polymer lowers the glass transition temperature (Tg).

In another embodiment, the two types of micellar crosslinking polymers are lauryl acrylate and octadecyl acrylate.

In one embodiment, the hydrogel further includes a water trapper capable of hydrogen bonding, such as glycerol.

The glycerol may be introduced via solvent exchange.

The invention further provides an electrode comprising the above hydrogel and a metal formed thereon.

In one embodiment, the metal penetrates the hydrogel and is deposited by electroplating.

The electrode may have a gauge factor ranging from 0 to 0.004.

The invention also provides a biosensor comprising such an electrode.

According to the present invention, the use of nanoscale hydrophobic micelles introduces repulsive forces that prevent water or ion penetration, thereby imparting anti-swelling characteristics. Solvent exchange with a glycerol-water solution enables strong hydrogen bonding that prevents drying.

Furthermore, external stresses are distributed across the micelles and hydrogel, enhancing mechanical durability and ensuring a low gauge factor in gold electrodes. This ensures signal stability even under dynamic conditions, making the invention suitable for various biomedical device applications.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a schematic diagram showing the preparation of a stretchable, biocompatible electrode comprising an anti-swelling and anti-drying hydrogel with zero gauge factor characteristics.

FIG. 2 shows the analysis of the properties of the prepared hydrogel.

FIG. 3 compares the hydrogel properties with conventional technologies.

FIG. 4 shows evaluation results of zero gauge factor in hydrogel-based electrodes.

FIG. 5 shows the depth profile concentration change of gold clusters deposited on polymer and hydrogel under strain.

FIG. 6 shows the adhesion properties of the anti-swelling and anti-drying hydrogel.

FIG. 7 illustrates application examples of the zero gauge factor electrode in bio signal sensing.

In the following description, the same or similar elements are labeled with the same or similar reference numbers.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In addition, a term such as a “unit”, a “module”, a “block” or like, when used in the specification, represents a unit that processes at least one function or operation, and the unit or the like may be implemented by hardware or software or a combination of hardware and software.

Reference herein to a layer formed “on” a substrate or other layer refers to a layer formed directly on top of the substrate or other layer or to an intermediate layer or intermediate layers formed on the substrate or other layer. It will also be understood by those skilled in the art that structures or shapes that are “adjacent” to other structures or shapes may have portions that overlap or are disposed below the adjacent features.

In this specification, the relative terms, such as “below”, “above”, “upper”, “lower”, “horizontal”, and “vertical”, may be used to describe the relationship of one component, layer, or region to another component, layer, or region, as shown in the accompanying drawings. It is to be understood that these terms are intended to encompass not only the directions indicated in the figures, but also the other directions of the elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Preferred embodiments will now be described more fully hereinafter with reference to the accompanying drawings. However, they may be embodied in different forms and should not be construed as limited to the embodiments set forth herein. Rather, these embodiments are provided so that this disclosure will be thorough and complete, and will fully convey the scope of the disclosure to those skilled in the art.

The present invention provides a hydrogel and an electrode based thereon that can measure stable biological signals despite the stretchability and deformation of the attached body, in order to solve the above-mentioned problems.

To this end, unlike conventional technologies that produce a non-swelling hydrogel by coating the hydrogel surface with a hydrophobic polymer or blending it with a hydrophobic polymer, the present invention applies a hydrophobic micelle synthesis technology at the nanometer scale that can induce repulsion to water or ionic components penetrating into the hydrogel. This effectively solves the problems of polymer coating methods that require high temperatures and long heat treatment times, significantly increase the Young's modulus of the hydrogel, and reduce adhesion to skin and biological tissues, and hydrophobic polymer blending methods that have limitations in preserving the physical properties of the hydrogel due to polymer aggregation when exposed to water for long periods.

That is, the present invention solves the problem of hydrogel swelling and drying through the introduction of hydrophobic micelles and solvent exchange technology, and in particular, formed micelle-crosslinked polymers within the hydrogel substrate using multiple types of acrylate oil emulsions with different chain lengths.

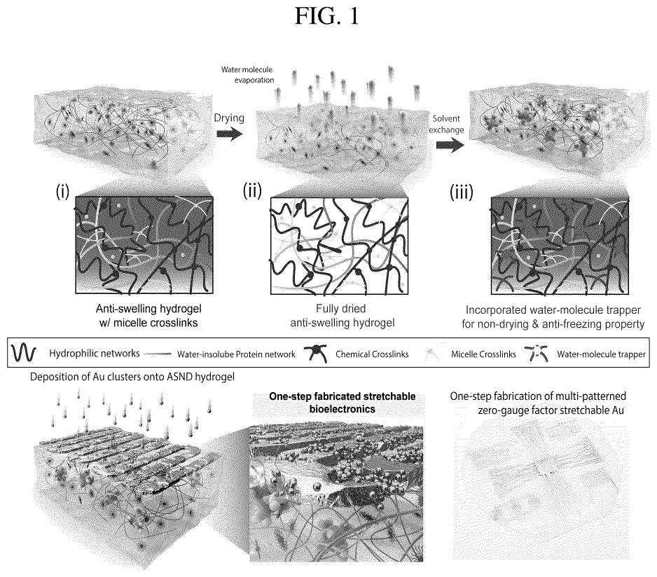

FIG. 1 is a schematic diagram of a method for manufacturing a stretchable biocompatible electrode material including an anti-swelling and anti-dry hydrogel that imparts zero gauge factor characteristics according to an embodiment of the present invention.

Referring to FIG. 1, the anti-swelling-anti-dry hydrogel includes a hydrogel substrate; and two types of micelle-crosslinked polymers (yellow chains) having different lengths, formed within the hydrogel substrate. In addition, it includes a water trapper (glycerol as red chains in FIG. 1), which is a substance capable of hydrogen bonding within the hydrogel to trap water, and the present invention will be described in more detail through the following examples.

Also, the micellar network within the hydrogel matrix, as illustrated in FIG. 1, comprises two types of hydrophobic acrylates (lauryl acrylate and octadecyl acrylate) optimally blended at a weight ratio of 40:60 to 60:40, preferably 50:50. These nanoscale micelles (approximately 10-100 nm) form a uniform dispersion, ensuring balanced elasticity and structural stability.

Examples

The anti-swelling hydrogel is prepared as follows. First, Sodium dodecyl sulfate (SDS) was dissolved in DI water to a concentration of 7 w/v %, and then Zein, a protein-based adhesive material insoluble in water, was added to a concentration of 10 w/v %.

Zein was used because it can preserve adhesion to tissues or skin by preventing the deterioration of adhesion properties even in a humid environment.

SDS was added to DI water to adjust the pH to dissolve such protein material. In addition, it was used to develop a hydrophobic oil emulsion complex into a micelle structure.

This mixture was stirred on a hot plate at 65° C. and 250 rpm for 24 hours. Thereafter, 0.3 mL of a micelle solution was added to this mixture.

In one embodiment of the present invention, the materials constituting the micelle solution were two types of acrylates with different chain lengths, one of which was lauryl acrylate and the other was octadecyl acrylate.

That is, in one embodiment of the present invention, lauryl acrylate used has a short alkyl chain, which has the disadvantage of a small hydrophobic effect. Octadecyl acrylate has a relatively long chain and is strongly hydrophobic, but has the disadvantage of a high glass transition temperature (Tg) (48 degrees), making it difficult to use as it precipitates when used in an atmospheric environment. Therefore, the present invention used a complex solution that can be uniformly mixed with the hydrogel as a micelle complex solution by lowering Tg using lauryl acrylate, which has a short chain length, among two types of micelle-crosslinked polymers.

In one embodiment of the present invention, the weight ratio of lauryl acrylate to octadecyl acrylate was 5:5.

Thereafter, the completely dissolved hydrophobic solution was transferred to a new vial and mixed with DI water (25 v/v %).

Subsequently, acrylamide (44 w/v %), N,N′-Methylenebis(acrylamide) (99%) (MBA) (0.038 w/v %), and α-ketoglutaric acid (0.2 w/v %) were sequentially added, and then stirred at 65° C. and 250 rpm until all components were completely dissolved.

Finally, agarose (7 w/v %) was added to the solution and stirred at 350 rpm while heating to 92° C. until completely dissolved.

Acrylamide and agarose were used as hydrogel monomers, MBA as a crosslinking agent, and α-ketoglutaric acid as a photo-initiator.

A hydrogel solution (55 w/v %) was poured into a 90×15 mm petri dish, and then a hydrogel substrate was prepared by photopolymerization using a UV lamp (LGA10100F, Liim Tech, 365 nm, 900-1000 mW/cm², intensity 80%, 30 seconds).

The photopolymerized hydrogel substrate was dried in a vacuum oven (OV3-30, JEIO-TECH) at 45° C. under a vacuum of 10−6 Pa for 30 minutes to evaporate all water molecules inside the hydrogel substrate.

Thereafter, it was immersed in a mixed solution of glycerol and DI-water (weight ratio 4:1) for one day to synthesize an anti-dry hydrogel through solvent exchange.

This was used to prevent the hydrogel from drying out, as glycerol acts as a trapper that induces strong hydrogen bonding to trap water inside the hydrogel.

Subsequently, to pattern electrodes on the anti-swelling and anti-dry hydrogel, Ti/Au (10 nm/200 nm) electrodes were deposited on the hydrogel based on an electrodeposition method and used as an electrode system.

Before depositing the gold electrodes, surface treatment was performed to improve the adhesion between the hydrogel and the gold clusters. For surface treatment, 0.1 mL-1.0 mL of APTES ((3-Aminopropyl)triethoxysilane) solution was dispersed on the hydrogel surface and surface-treated by a wet coating method for 24 hours.

Thereafter, the surface was cleaned with ethanol and DI water to ensure no residue remained on the hydrogel surface.

### Experiment

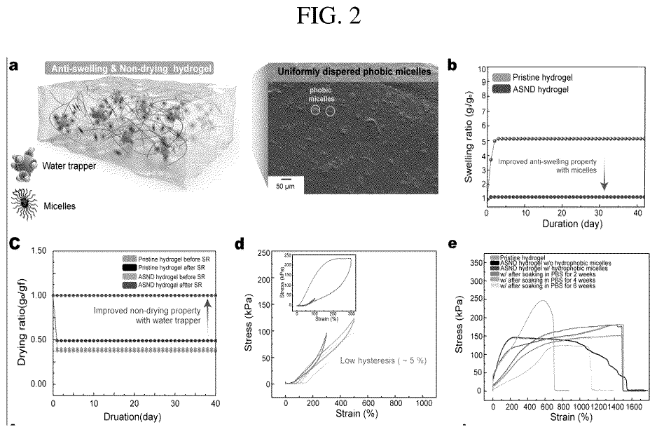

FIG. 2 shows a scanning electron microscope image result indicating that hydrophobic micelles are uniformly dispersed on the anti-swelling and anti-dry hydrogel.

Referring to FIG. 2, the micelles maintain a zeta potential lower than −60 mV, indicating electrostatic stability that prevents aggregation and promotes uniform integration into the hydrogel. This configuration mitigates localized stress concentration and enhances electrical uniformity.

Typically, the zeta potential value of a hydrophobic solution containing micelles shows a value higher than −60 mV, which prevents micelles from aggregating with each other and being uniformly dispersed within the hydrogel, thereby preventing the penetration of water or ion molecules from the outside through repulsive Coulomb forces generated from the micelles, thus solving the problem of structural change due to dilution of the hydrophilic network inside the hydrogel. This confirmed that when analyzing the swelling degree of a general hydrogel without micelles and the hydrogel of the present invention containing micelles, the swelling degree of the anti-swelling hydrogel (~20%) decreased compared to a general hydrogel (swelling degree: >~500%) for at least 6 weeks (in-vitro accelerated aging test at 45 degrees).

In addition, when the drying degree of the hydrogel was analyzed in a 45-degree chamber, the hydrogel to which micelle and solvent exchange technology was applied showed almost no drying (~0%) for more than 6 months, while a general hydrogel dried within several hours.

Furthermore, the uniform dispersion of micelles inside the hydrogel resulted in approximately 3 times higher stretchability (:>~1600%) compared to a general hydrogel. This is because the Young's modulus of the micelles is several MPa and that of the hydrogel is tens of kPa, so when initial external stress is applied, it is uniformly dispersed in the micelles and then the stress is divided into the hydrogel, thereby also improving mechanical properties.

In addition, it was confirmed that the mechanical properties of the anti-swelling and anti-dry hydrogel were maintained even after long-term immersion in PBS solution at 45 degrees.

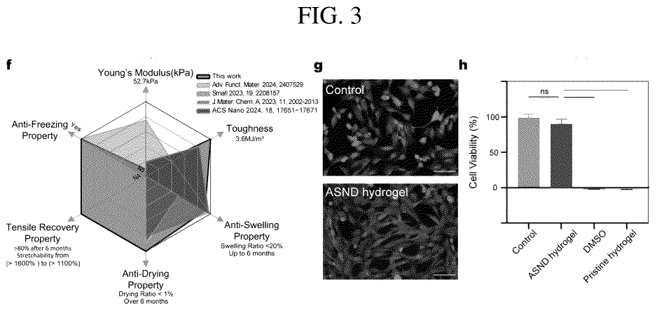

FIG. 3 shows the comparison results between the hydrogel of the present invention and hydrogels developed in prior art literature.

Referring to FIG. 3, the hydrogel demonstrates a swelling ratio of less than 25% and water loss under 5% after 6 months at 45° C., significantly outperforming conventional hydrogels that exhibit over 500% swelling and dry out within hours.

Furthermore, when the cytotoxicity of the anti-swelling and anti-dry hydrogel of the present invention was analyzed using 3T3 cells, one of the mouse embryonic fibroblast cell lines, it was confirmed that the hydrogel of the present invention had no cytotoxicity.

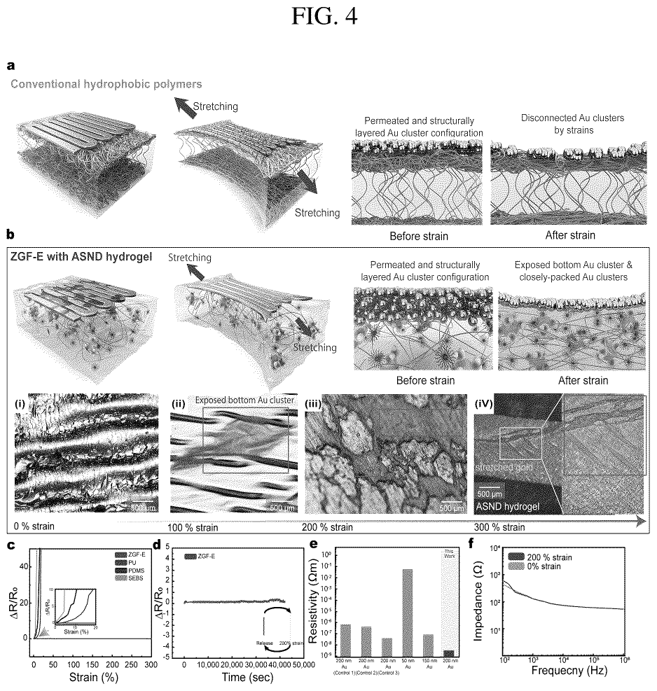

FIG. 4 shows the evaluation results of the zero gauge factor characteristics of the hydrogel-based electrode according to an embodiment of the present invention.

Referring to FIG. 4, the hydrogel-based electrode maintains a gauge factor in the range of 0.001 to 0.004 even after repeated deformation, with resistance variation of less than 2% after 1,000 strain cycles. This stability under strain supports its suitability for reliable biosignal acquisition. In the case of general hydrophobic polymers, when gold is electrodeposited, the connection between gold clusters cannot be maintained when external stress is applied, so the characteristics of the electrode cannot be maintained. However, when gold clusters are deposited on the anti-swelling and anti-dry hydrogel of the present invention, the gold clusters penetrate into the hydrogel, forming a soft-rigid island interface between the hydrogel and the gold clusters, with gold clusters having a Young's modulus of ~80 GPa strongly bonded from the hydrogel surface to the deep region. That is, the penetration occurs at least several nanometers or more from the surface to the inside, thereby enabling the implementation of a system where the penetrated gold clusters rise to the surface layer when stress is applied, increasing electrical conductivity and keeping the resistance unchanged, resulting in a gauge factor of 0 (more specifically, 0 to 0.004, which is near 0). This explains the phenomenon that when external stress is applied, the stress is dispersed to the micelles and the hydrogel frame, preventing deformation of the gold clusters, and the gold clusters that have penetrated into the deep region rise to the surface layer, increasing electrical conductivity and keeping the resistance unchanged.

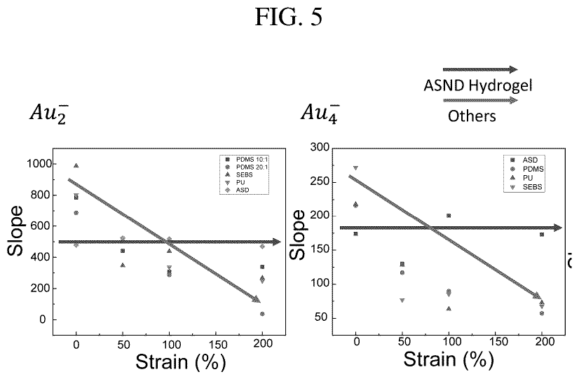

FIG. 5 shows the analysis results of the change rate of gold clusters deposited on a polymer and hydrogel according to depth due to elongation.

Referring to FIG. 5, TOF-SIMS analysis reveals that the concentration of gold clusters embedded in the hydrogel varies by less than 10% under mechanical strain, and the clusters penetrate to depths of several tens of nanometers, forming a stable conductive path.

Also, TOF-SIMS was used to analyze the change in gold clusters according to the depth of the hydrophobic polymer and anti-swelling and anti-dry hydrogel. While the concentration of gold clusters decreased when stretched in the case of general hydrophobic polymers, it was confirmed that there was no change in the concentration of gold clusters when stretched in the case of anti-swelling and anti-dry hydrogels. This explains the phenomenon that even under external stress, the concentration of gold clusters deposited on the anti-swelling and anti-dry hydrogel is maintained as the gold clusters that have penetrated deeply rise to the upper layer, thereby maintaining resistance.

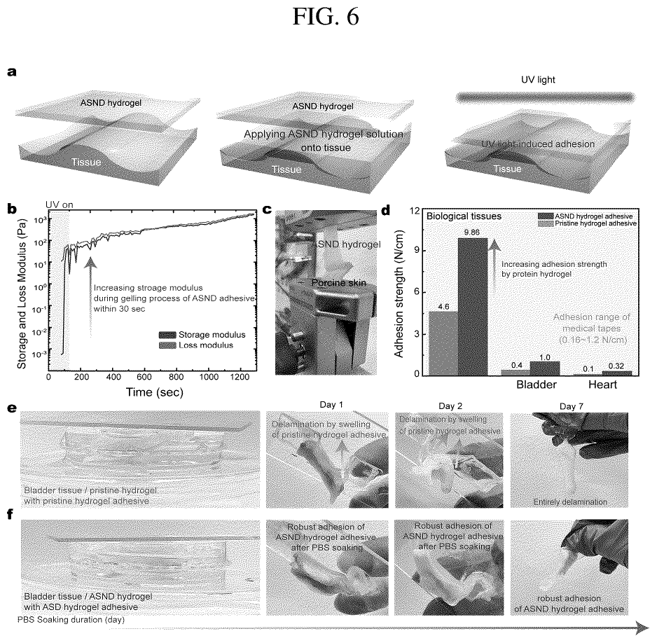

FIG. 6 shows the adhesion property evaluation of the anti-swelling and anti-dry hydrogel.

Referring to FIG. 6, the UV-cured hydrogel adheres to tissue surfaces with initial adhesion strength exceeding 0.5 N/cm² and maintains strong attachment for at least 7 days in PBS at 45° C., distinguishing it from typical adhesives.

The adhesion was confirmed through the hydrogel solution of the present invention. To attach the electrode system having a zero gauge factor to the tissue surface, a portion of the anti-swelling hydrogel solution was dispersed on the tissue surface, and when UV light was applied through a UV flashlight lamp, the anti-swelling hydrogel solution underwent photopolymerization within 30 seconds, thereby inducing adhesion between the electrode system and the tissue surface. It was confirmed that the adhesive material thus developed maintained its adhesion properties even after long-term immersion in PBS, unlike general hydrogel adhesive materials, due to its anti-swelling properties.

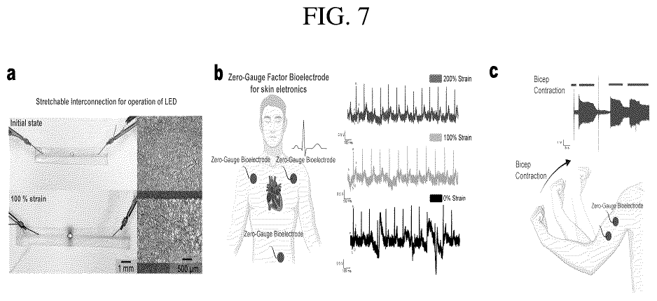

FIG. 7 is a diagram showing the applicability of the zero gauge factor gold electrode. Referring to FIG. 7, it can be seen that the hydrogel-based electrode material according to the present invention can be used in various biological activity information measuring devices such as heart rate measurement or muscle strength measurement. In more detail, Referring to FIG. 7, the electrode achieves accurate biosignal detection including EMG and ECG with signal-to-noise ratios exceeding 30 dB and measurement errors within ±5%, supporting its applicability in wearable and implantable health monitoring devices.

While the present disclosure has been described with reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims.

The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. An anti-swelling and anti-drying hydrogel comprising:
a hydrogel matrix; and
two types of micelle-crosslinked polymers formed within the hydrogel matrix,
wherein the two types of micelle-crosslinked polymers have different chain lengths.

2. The hydrogel of claim 1, wherein the micelle-crosslinked polymer with the shortest chain length functions to lower the glass transition temperature (Tg) of the micelle-crosslinked polymers.

3. The hydrogel of claim 1, wherein the two types of micelle-crosslinked polymers are lauryl acrylate and octadecyl acrylate, respectively.

4. The hydrogel of claim 1, further comprising a water trapper capable of forming hydrogen bonds within the hydrogel matrix.

5. The hydrogel of claim 4, wherein the water trapper is glycerol.

6. The hydrogel of claim 5, wherein the glycerol is introduced into the hydrogel via a solvent exchange process.

7. An electrode comprising:
the hydrogel of claim 1; and
a metal formed on the hydrogel.

8. The electrode of claim 7, wherein the metal is formed by penetrating into the hydrogel.

9. The electrode of claim 8, wherein the metal is deposited into the hydrogel via electroplating.

10. The electrode of claim 9, wherein the electrode has a gauge factor in the range of 0 to 0.004.

11. A biosensor comprising the electrode of claim 10.

12. The hydrogel of claim 2, further comprising a water trapper capable of forming hydrogen bonds within the hydrogel matrix.

13. The hydrogel of claim 12, wherein the water trapper is glycerol.

14. The hydrogel of claim 13, wherein the glycerol is introduced into the hydrogel via a solvent exchange process.

15. The hydrogel of claim 3, further comprising a water trapper capable of forming hydrogen bonds within the hydrogel matrix.

16. The hydrogel of claim 15, wherein the water trapper is glycerol.

17. The hydrogel of claim 16, wherein the glycerol is introduced into the hydrogel via a solvent exchange process.
